(19) United States
(12) Patent Application Publication
(10) Pub. No.: US 2026/0124595 A1
(43) Pub. Date: May 7, 2026

(54) UREA PLANT WITH MODIFIED CONDENSER AND MODIFIED REACTOR INTERNALS

(71) Applicant: Stamicarbon B.V., Sittard (NL)

(72) Inventors: Wilhelmus Hubertus GEURTS, Born (NL); Paul Wilhelmus Maria MEIJBOOM, Gulpen (NL)

(73) Assignee: Stamicarbon B.V., Sittard (NL)

(21) Appl. No.: 19/480,616

(22) PCT Filed: Mar. 20, 2025
(86) PCT No.: PCT/NL2025/050138

§ 371 Date: Oct. 31, 2025

(30) Foreign Application Priority Data
Mar. 20, 2024 (EP) 24164925.0
Dec. 18, 2024 (EP) 24221297.5

(51) Int. Cl. B01J 19/00; B01J 4/00; C07C 209/16

(52) U.S. Cl. CPC B01J 19/006; B01J 4/001; C07C 209/16; B01J 2219/00024; B01J 2219/00763

(57) ABSTRACT

The disclosure pertains to a modification of a stripping-type urea plant by replacing the falling-film type HP carbamate condenser with a horizontal submerged condenser in combination with modification of the reactor internals, thereby permitting to maintain gravity flow in the modified plant.

### FIELD

The invention pertains to the production of urea.

### INTRODUCTION

A frequently used type of urea production process is the so-called stripping process, wherein the high-pressure urea synthesis section comprises a reaction zone, a stripper, and a condenser. A background reference for such processes is Ullmann's Encyclopaedia of Industrial Chemistry, chapter Urea, 2010. This document mentions that some processes use a recycle flow purely based on gravity, i.e. to maintain the main recycle flow in the high-pressure (HP) loop of the synthesis section, formed by the flow from the stripper to the condenser, from the condenser to the reactor, and from the reactor to the stripper.

The invention is particularly directed to the CO₂ stripping type urea production process, wherein the urea solution from the reactor is stripped with feed CO₂ gas in the stripper comprised in the high-pressure section.

An early embodiment of the CO₂ stripping type urea production process and plant is described in Kaasenbrood and Chermin, “The Urea Stripping Process”, Fertiliser Society, 1977. In the embodiment described therein, gas from the top of HP stripper flows to the head of the carbamate condenser. In the condenser tube the gas is partly condensed. The liquid-gas mixture leaving the condenser is introduced into the bottom of the reactor. Reactor, stripper, and condenser have a spatial arrangement such that the driving flow through these vessels is provided by variations in phase and hence fluid density, brought about by the supply of heat in the stripper and the withdrawal of heat in the condenser. The top of the stripper is about 10 m below the bottom of the reactor and the condenser. The reactor is operated adiabatically, which is realised by the condensation of the gas mixture from the condenser. FIG. 7 of Kaasenbrood 1977 shows that the condenser in that embodiment is a vertical shell-and-tube heat exchanger with steam raised in the shell and with gas from the stripper supplied to the top of the tube bundle and with an outlet at the bottom for supplying fluid to the bottom of the vertical reactor. The bottom of the reactor and of the condenser are at equal height. Liquid is withdrawn from the top of the reactor and supplied to the liquid inlet of the upper chamber of the stripper.

An improved type of high-pressure condenser is described in EP0155735A1. Herein gas to be condensed is introduced to the shell-side of a shell-and-tube heat exchanger with a horizontal tube bundle, in particular with a U-shaped tube bundle, wherein in operation continuous liquid phase is maintained in the shell side compartment. Thereby the condenser operates as a horizontal submerged condenser. The bottom of the vertical reactor is shown as substantially the same height as the fluid outlet of the pool condenser, which is located in a top part of the pool condenser.

It is often desired to increase the capacity of existing urea plants, making maximum use of the existing equipment and with minimum modifications. Such projects are known as ‘revamps’ in the art. Furthermore, a number of existing urea plants of the CO₂ stripping type with a falling-film high-pressure carbamate condenser are still operating. The present invention in an aspect aims to provide a revamp method for such urea plants.

EP1036787A1 discusses a revamping method wherein a falling-film type condenser of the HP synthesis section is modified into a vertical submerged condenser. A further reference for such a method is US20080242890A1.

US2015343409A1 mentions as a known way of increasing the reactor volume of an existing plant, to replace “the HPCC” (high-pressure carbamate condenser) with a pool condenser. It is said that this requires a complicated equipment design. The document proposes to increase by installing an additional reactor.

US20030088126A1 FIG. 4B shows a plant with a pool condenser, with fluid from the condenser being supplied to the bottom of an upper compartment of a vertical combination reactor. Gas from the bottom compartment of the vertical combination reactor is released into the upper compartment. An ejector is used to supply a part of the gas from the stripper to the bottom compartment of the vertical combination reactor.

EP0329215A1 shows a urea process with a HP CO₂ stripper, a vertical reactor, and a condenser. Liquid from the condenser is supplied to the bottom of the reactor, gas from the condenser is supplied to a HP scrubber. A part of the gas from the stripper is supplied to the bottom of the reactor using an ejector driven by a part of the ammonia feed. Gas from the reactor top is supplied to the condenser. The document explains that if the line connecting the condenser with the reactor as well as the line from the rector to the stripper are filled with liquid, these equipment items function as communicating vessels, enabling liquid transport by gravity from the condensation zone through the reaction zone to the stripping zone. Non-condensed gas from the horizontal carbamate condenser is supplied to a HP scrubber. The condenser is a horizontally mounted carbamate-condenser, situated 20 meter above ground level, with a submerged condenser design and a diameter of about 3 metres. The reactor is placed at ground level and has a height of about 18 meters. The stripper is placed at ground level with a height of about 11 meters.

US20110160486A1 describes a urea production process wherein the process runs totally on gravity flow for the main recycle of non-converted ammonia and carbon dioxide in the high-pressure synthesis section of the urea production process, such that the use of energy consuming pumps, compressors or ejectors is superfluous.

### SUMMARY

The invention provides in an embodiment a method of modifying an existing urea plant, wherein the existing urea plant comprises a high-pressure synthesis section comprising: a vertical urea reactor; a stripper having an inlet for gaseous CO₂ feed; a high-pressure carbamate condenser configured to receive gas from the stripper, wherein the high-pressure carbamate condenser is a falling-film shell-and-tube heat exchanger having a bottom outlet from the tubes connected to a bottom inlet of the reactor, wherein the reactor comprises one or more feed pipes for fluid from the condenser, the one or more feed pipes having an outlet at a first vertical level in the reactor; wherein the method involves: replacing the high-pressure carbamate condenser by a replacement carbamate condenser that is a horizontal submerged condenser which is a shell-and-tube heat exchanger with condensation in the shell side space, preferably wherein the replacement condenser is a pool condenser; and modifying at least one of the one or more feed pipes of the reactor to have an outlet at a second vertical level that is higher than the first vertical level. Preferably, the reactor comprises a first feed pipe to receive gas from the condenser and a second feed pipe to receive liquid from the condenser both before and after the modification, and the method involves replacing the carbamate condenser with the horizontal submerged condenser, and extending the first feed pipe for gas to have an outlet at said second vertical level. In an alternative embodiment, the existing reactor (before the modification) contains no feed pipes and the modification involves adding one or more of such feed pipes for fluid from the condenser. Preferences for the modified feed pipes, in particular the second vertical level, apply also to the added one or more feed pipes.

The second vertical level is preferably at least 1.0 m higher or at least 2.0 m higher than the first vertical level. The extended feed pipe(s) is (are) extended preferably by at least 1.0 m or at least 2.0 m. The first vertical level is e.g. less than 0.50 m from the bottom of the reactor.

Also provided is a urea plant comprising a high-pressure synthesis section comprising: a vertical urea reactor comprising a first and a second feed pipe; a stripper having an inlet for gaseous CO₂ feed; a high-pressure (HP) carbamate condenser configured to receive gas from the stripper, wherein the high-pressure carbamate condenser is a horizontal submerged shell-and-tube heat exchanger with condensation of gas from the stripper in the shell, having a horizontal tube bundle, preferably wherein the HP carbamate condenser is a pool condenser, and a liquid flow line for condensate from the condenser to a first feed pipe of the reactor and a separate gas flow line from the condenser to a second feed pipe of the reactor; wherein the vertical distance between the outlet of the first feed pipe and/or the outlet of the second feed pipe to the bottom of the reactor is preferably at least equal to the height (i.e. diameter) of said tube bundle, or, more preferably, at least equal to the height or diameter of the shell of the pool condenser.

The plant is preferably obtained by the inventive modification of an existing plant.

Also provided is a urea production process carried out in a plant according to the invention, the process comprising supplying CO₂ feed to said stripper and NH₃ to said high-pressure carbamate condenser, and operating the high-pressure synthesis section under a pressure of at least 100 bar to form urea.

### BRIEF DESCRIPTION OF THE DRAWINGS

FIG. 1 schematically illustrates an example existing urea plant.

FIG. 2 schematically illustrates an example inventive urea plant.

FIG. 3 schematically illustrates a part of an example reactor of an inventive urea plant.

Any embodiments illustrated in the figures are examples only and do not limit the invention.

### DETAILED DESCRIPTION

The invention advantageously provides, in an aspect, a method of modifying an existing urea plant by introducing a horizontal submerged condenser while advantageously maintaining gravity flow in the synthesis section. It is an insight of the present inventors that this can be done with a suitable modification of a feed pipes in the reactor, in preferred embodiments of the invention.

The invention provides, in an embodiment, a method of modifying an existing urea plant. Briefly, the existing urea plant is of the CO₂ stripping type and comprises a high-pressure synthesis section comprising a reactor, a condenser and a stripper. The existing condenser is a falling film HP carbamate condenser (HPCC). The plant is modified by replacing the falling-film HPCC with a horizontal submerged condenser, preferably a pool condenser. A pool condenser comprises one or more U-shaped tube bundles. An example design of a pool condenser is shown in Nitrogen No. 222 (July-August 1996), p. 29. Optionally, the pool condenser comprises an extended spacing between the bend of the U-shaped tube bundle and the shell to provide additional reactor volume. In operation, the U-tube bundle is submerged in the liquid present and formed in the shell side space. The U-shaped tube bundle has horizontal legs. The horizontal submerged condenser comprises e.g. a sparger for distributing gas from the stripper in the shell-side space. The horizontal submerged condenser is generally a shell-and-tube heat exchanger with a tube bundle, a shell, and a shell-side space, configured to receive gas from the stripper in the shell side space and cooling fluid, e.g. boiler feed water, in the tubes.

The pool condenser provides the advantage of a urea production capacity increase of the plant by the higher residence time of the carbamate solution formed as condensate in the pool condenser compared to the falling-film type HPCC. Hence, the urea production (ton urea per hour) can be increased without modification of the HP stripper.

The degree of condensation in the pool condenser can be adjusted as necessary, e.g. by adjusting the operating pressure of steam produced in the tube bundle. Hence, sufficient non-condensed gas can remain to provide heat for the reactor.

A liquid layer is maintained in the process side of pool condenser, i.e. in the shell side space of the pool condenser, unlike in a falling film type HPCC. In the pool condenser, the gas from the stripper is supplied into the shell from the bottom and the fluid exit of the shell is at the top, such that the liquid layer imparts a pressure drop. Therefore, with a fixed pressure of the gas from the stripper, the gas and liquid from the pool condenser has a lower pressure. The invention advantageously provides for maintaining fluid flow from the condenser to the reactor and liquid flow from the reactor to the stripper, in particular advantageously based on gravity flow. In the existing plant, the liquid flow from the condenser, through the reactor, to the stripper, is maintained by ‘gravity flow’, i.e. by making use of the position of the condenser at a higher level than the top of the stripper in the plant, without the use of pumps or ejectors. Furthermore, in the existing plant, non-condensed gas from the condenser is supplied to the bottom to the reactor. This provides the advantage that the condensation of CO₂ and NH₃ comprised in the non-condensed gas into carbamate in the reactor is used to provide heat to drive the endothermic urea-formation reaction (dehydration of carbamate) in the reactor. The invention provides the advantage of supplying non-condensed gas from the pool condenser to the reactor in the modified plant to provide heat to the reactor. The invention very advantageously provides for gravity flow in the modified plant.

In the existing plant, the urea reactor is a vertical urea reactor, with a feed inlet at the bottom and with a downpipe for urea solution that is withdrawn from an upper part of the reactor. The downpipe is also known as an overflow pipe and as a downcomer. The upper end of the downpipe acts as an overflow weir in operation, with the liquid level in the reactor at said upper end. In operation, a liquid column is maintained in the downpipe.

The downpipe usually has a funnel at the upper end for degassing the urea solution. In operation, a liquid level is maintained in the urea plant at the height of the upper end of the downpipe. The reactor has a separate gas outlet at the top. The reactor after the modification preferably also has these features.

Supplying non-condensed gas from the horizontal submerged condenser to the reactor, in the invention, provides advantages, e.g. compared to an example reference process wherein heat is provided to the reactor by supplying a part of the feed CO₂ gas directly to the reactor instead of to the stripper, as this reference process has a relatively lower efficacy of the HP stripper (lower stripping efficiency) and may lead to excess NH₃ in the condenser. A reference process involving supplying a part of the gas from the stripper directly to the reactor involves challenges in dividing the gas stream, can be detrimental to gravity flow if the gas condenses at low elevation in the reactor, and may require the use of an ejector in view of the large liquid column in the reactor.

In the existing plant, the synthesis section comprises for example a high-pressure scrubber. In the HP scrubber, off-gas from the top gas outlet of the reactor, comprising NH₃, CO₂ and inerts, is scrubbed to recover the CO₂ and NH₃ as carbamate solution. The inert components include typically passivation air and includes non-condensable gases. The aqueous scrub liquid in the HP scrubber is typically the recycle carbamate solution from the recovery section and water contained in it also serves as solvent in the HP carbamate condenser. For example, carbamate solution from the HP scrubber is supplied to the falling-film carbamate condenser in the existing plant using an ammonia-driven ejector, and for example also in the modified plant. For example, the existing plant operates with an oxygen content of about 0.6% (e.g. at least 0.3% by volume O₂ in the CO₂ feed) to sufficiently passivate the high-pressure synthesis section, e.g. any parts made of urea grade stainless steel thereof. For example, the modified plant also operates with passivation air added to the CO₂ feed in an amount of least 0.3 vol. % O₂, relative to CO₂ feed.

In the prior art urea production process with a pool condenser illustrated in FIG. 22 of Ullmann's Urea 2010, the so-called Avancore® process, only the carbamate solution from the pool condenser is supplied to the bottom of the reactor placed at ground level, with a liquid column in the flow line. The gas from the condenser is supplied to a medium pressure scrubber. A part of the CO₂ feed is supplied to the bottom of the vertical reactor. Furthermore, in the prior art urea production process of FIG. 28 of Ullmann's Urea 2010, the ACES21 process, a vertical submerged condenser is used, with carbamate solution being supplied to the bottom of the reactor by an ejector using feed NH₃ and with gas from the condenser supplied to a medium pressure absorption unit. A part of the CO₂ feed is supplied to the reactor.

In a preferred embodiment of the invention, the method involves a modification of the internals of the reactor, advantageously without any sealing of a part of the reactor.

Preferably, before the modification, the vertical urea reactor comprises one or more feed pipes for gas and liquid from the HP carbamate condenser into the reactor space at a first vertical level in the reactor. Herein, the term ‘feed pipe’ is used broadly and can include tubes and ducts and can also be referred to as supply means. In this embodiment, the existing feed pipes are modified as part of the modification.

In an alternative embodiment, the existing reactor has one or more inlets for gas and liquid from the existing falling-film HP carbamate condenser, but no feed pipes inside the reactor, and one or more feed pipes are added as part of the modification. Preferences for the modified feed pipes apply also the added feed pipes. Preferably, any added gas feed pipe has a length of at least 1.0 m or at least 2.0 m. This alternative embodiment can conveniently be used in case the existing vertical urea reactor has one inlet openings at the bottom for fluid from the HP carbamate condenser but no feed pipes that extend (vertically) into the reactor space for such fluids.

Preferably, the reactor comprises a feed pipe for liquid and a (separate) feed pipe for gas (in particular, before and after the modification) from the HP carbamate condenser; preferably the plant comprises separate flow lines for gas and for liquid from the condenser to the reactor (in particular, before and after the modification), and preferably the condenser before and after modification comprises a gas/liquid separator. The gas/liquid separator is e.g. an overflow weir, e.g. provided by an outlet opening in the wall of a vertical upward outlet pipe mounted on the upper side of the pool condenser, which pipe has appropriate gas outlet openings at the top.

The feed pipe for the liquid is e.g. a straight vertical pipe with upper outlet end. The feed pipe for the gas is e.g. a pipe with an outlet end that is bent downwards (gooseneck piping).

The feed pipe for liquid can be relatively short, extending only slightly from the reactor bottom wall, which is e.g. a hemispherical head.

In the highly preferred modification, at least the feed pipe for gas is modified to introduce gas from the HP carbamate condenser at a second vertical level in the reactor, wherein the second vertical level is higher than the first vertical level. Herein, the vertical level refers to the distance between the bottom of the reactor and the point of introduction of the fluid. Hence, the vertical distance between the outlet end of the feed pipe for gas and the reactor bottom is increased by the modification. The second vertical level is usually provided (defined) by the open end of the gas feed pipe. Preferably the gas feed pipe is extended to have the outlet at a higher level in the reactor, e.g. by at least 1.0 m or least 2.0 m.

It is an insight of the present inventors that in the modified plant, the gas is introduced, from the extended gas feed pipe, into a shorter liquid column in the reactor, namely at a shorter vertical distance to the liquid level (maintained at an upper part of the reactor at the inlet of the downpipe) and hence at a lower pressure, than when introduced at the first vertical level (as before the modification) and that this pressure reduction very advantageously may contribute to keeping gravity flow operation in the modified plant by compensating for the pressure of the liquid level maintained in the horizontal submerged condenser.

Preferably, the feed pipe for liquid is also extended. Preferably, the feed pipes for gas and for liquid are modified to introduce gas and liquid from the HP carbamate condenser at a second vertical level in the reactor. Introducing gas and liquid at the same vertical level in the reactor may provide for a more robust process design.

An advantage of this embodiment, compared to a less preferred method that involves sealing off a bottom part of the reactor, is that also the bottom part of the reactor can be used in operation, i.e. the part of the reactor below the second vertical level, since the bottom part of the reactor will be filled with reaction medium in operation, in particular with liquid.

The bottom part of the modified reactor is the part between the bottom of the reactor and the second vertical level.

If gas and liquid are supplied from the horizontal submerged condenser (e.g. pool condenser) into the reactor with separate flow lines and feed pipes, both feed pipes preferably have an outlet at the same vertical level, i.e. the second vertical level. This preferred feature ensures equal pressure at the respective inlets of the flow lines in the horizontal submerged condenser, e.g. pool condenser. In another embodiment, the outlet of the gas feed pipe is at a higher vertical level than the outlet of the liquid feed pipe after the modification.

The height of the bottom part (and the distance between the bottom of the reactor and the second vertical level) is typically substantially the same as the diameter or height of the pool condenser, e.g. approx. 3 meters. In a preferred example embodiment, the gas feed pipe (and optionally the liquid feed pipe as well) is extended in the vertical direction by the height of the pool condenser. In particular, the vertical distance between the first and the second vertical level is the same as the diameter of the horizontal cylindrical vessel of the pool condenser. This preference applies especially if the cylindrical horizontal vessel of the pool condenser is filled with liquid as continuous phase in operation and liquid level is maintained in a dome mounted on top of the horizontal cylindrical vessel in operation. These preferences for the heights also apply to any added feed pipes in the embodiment involving adding feed pipes.

Preferably, the horizontal submerged shell-and-tube heat exchanger comprises a capped horizontal cylindrical vessel holding the horizontal tube bundle. The vessel can be capped, e.g. with a hemi-head cap at one end and a tube sheet at another end. Preferably, the vertical distance (h2) between the outlet of the second feed pipe for gas to the bottom of the reactor is equal to or larger than the diameter of said horizontal cylindrical vessel. This embodiment is preferably used with a dome for maintaining a liquid level on the upper side of the horizontal vessel, the dome having a liquid outlet and a separate gas outlet, these outlets connected to the liquid feed pipe and the gas feed pipe, respectively. The gas and liquid outlets of the dome are preferably vertically spaced from each other.

It is observed that especially for a pool condenser that is horizontal and has a length that is larger than the diameter of the reactor, the reactor volume provided by the pool condenser is larger than the reactor volume of this bottom part.

In a possible embodiment with the feed pipe modified to introduce fluid at the second vertical level, the part of the reactor below the second vertical level is lined with duplex stainless steel (i.e. duplex ferritic austenitic steel, for example but without limitation UNS S32906 steel). This may be useful since the liquid in the bottom part can be stagnant. Moreover, the liquid in the bottom part will contain carbamate, at relatively high temperature, and can therefore be highly corrosive.

In a more preferred embodiment, a fluid comprising a passivation agent, e.g. a fluid comprising O₂, is introduced into the bottom part. This preference applies to the urea production process; correspondingly the inventive urea production plant preferably comprises a reactor with a bottom part with an inlet for a fluid comprising O₂, more in particular a suitably flow line for a fluid comprising O₂ into said reactor bottom part.

In existing urea plants, passivation with an oxidizing species, usually O₂, is frequently used to avoid corrosion of the equipment in the high-pressure synthesis section that is in contact with carbamate-containing liquid in operation. Typically, some air is mixed into the CO₂ gaseous feed for these purposes. In a urea plant of the CO₂ stripping type, this serves for passivation of the stripper and condenser, and by the supply of gas from the condenser to the reactor, or by the supply of a part of the CO₂ feed or a part of the gas from the stripper to the reactor, also for passivation of the reactor.

The passivation agent is a fluid comprising O₂, e.g. a gas comprising O₂ or a liquid comprising dissolved O₂.

In a preferred embodiment, the passivation agent introduced into the bottom part, hence the O₂ containing fluid, is gaseous and is e.g. air, or a part of the CO₂ feed (comprising O₂), or a part of the gas from the stripper. Introducing gas from the stripper into the reactor will generally involve the need for an ejector.

In yet a further embodiment, the passivation agent is a liquid comprising dissolved O₂, e.g. a carbamate solution from a high-pressure scrubber. The plant preferably comprises a HP scrubber connected to receive off-gas from the reactor (top outlet of the reactor) and an aqueous scrub liquid. The scrubber is preferably arranged above the reactor and the scrubbing involves condensation of the gas into carbamate solution in operation. The off-gas from the reactor comprises NH₃, CO₂, and inerts, the inerts include O₂. The liquid comprising dissolved O₂ is preferably introduced into the bottom part of the reactor with a liquid distributor for distributing the gas horizontally, e.g. a sparger. Introducing the liquid comprising dissolved O₂ at the bottom of the reactor, can be used to prevent corrosion by stagnant liquid carbamate solution in the bottom compartment.

In a possible embodiment, lining the bottom part of the reactor with duplex stainless steel is combined with introducing a fluid comprising a passivation agent into to the bottom part of the reactor.

In a preferred embodiment, the method of modifying a urea plant involves providing the reactor with an inlet for an oxygen-containing fluid in a bottom reactor compartment that is provided between the reactor bottom and the second vertical level.

In a preferred embodiment, the reactor is modified (in the method of modifying an existing urea plant) by providing an additional inlet for an additional fluid and preferably also with internal supply means (e.g. a pipe, tube or duct) for introducing the additional fluid into the reactor at a third vertical level that is lower than the second vertical level. The third vertical level is hence located between the rector bottom and the second vertical level. The internal supply means are internal to the reactor. The first and third vertical level can be the same or different. Preferably, the third vertical level is close to the reactor bottom. Hence, the modified reactor comprises internal supply means for introducing additional fluid into the reactor vertically between the bottom of the reactor and the point of introduction of the gas and liquid from the pool condenser. This additional fluid contains a passivating agent, e.g. O₂, and can be used for passivating the reactor bottom part. This is especially advantageous if the reactor wall surface that is exposed to the carbamate solution consists of (e.g. is lined with) austenite steel (e.g. 316L steel, also known as EN 1.4404 and UNS S31603). The inventive urea production plant preferably comprises a reactor with such an inlet and internal supply means, and the urea production process is preferably carried out in a plant with such a reactor.

Moreover, preferably the additional fluid is a gas stream comprising CO₂ and the reaction of this gaseous CO₂ with NH₃ to form carbamate, that occurs in the bottom part of the reactor, also provides heat for the urea formation reaction in the bottom part of the reactor.

It is known in the art that stagnant carbamate-containing liquids involve an increased risk of corrosion by loss of passivation due to depletion of passivation air.

In a possible embodiment, the bottom part of the reactor is drained for liquid, e.g. to the stripper or to the ejector of the scrubber, with or without the introduction of passivating agent in the bottom part. By the purge of liquid, i.e. said drainage, a stagnant liquid in the bottom part is avoided, such that the corrosion risk is reduced. However, the conversion rate in the drained bottom part is not optimal.

In an embodiment, an existing pipe in the reactor is modified so as to be suitable for purging a fluid stream from the bottom part of the reactor.

For example, the existing downcomer of the reactor is modified with small perforations in the bottom part of the reactor. By the density difference of the fluid in the downcomer and in the bottom part of the reactor, liquid will flow from the bottom part into the downcomer.

In an embodiment, the reactor is modified by adding a pipe for purging a fluid stream from the bottom part of the reactor.

In a further preferred embodiment, liquid is made to move from the liquid feed pipe outlet (which is at the second vertical level, in this embodiment) to a vertical level below the second vertical level in the bottom part, in the urea production process carried out in the urea plant comprising the reactor. This provides the advantage that better use is made of the reaction volume provided by the bottom part, compared to substantially stagnant liquid in the bottom part. The modified reactor is preferably configured accordingly. For example, the bottom reactor part is provided with a vertical divider, preferably a tubular or annular vertical divider, to create a siphon flow, to create two vertical compartments in the bottom part that are substantially separated by the vertical divider, in particular a first compartment inside the vertical divider and a second compartment between the vertical divider and the reactor wall. The vertical divider separates these two compartments over a part of the length of the reactor, i.e. over a part of the reactor height, more in particular preferably over a height that is at least 80% or at least 90% of the height of the bottom compartment (i.e., the length of the divider is at least 80% or at least 90% of the distance between the reactor bottom and the second vertical level h₂). In operation, a siphon flow is obtained from the liquid feed pipe outlet downward into the bottom part through the second vertical compartment, and upward through the first vertical compartment (i.e. inside the tube, i.e. inside the tubular or annular vertical divider).

Preferably, gas is introduced preferentially into the bottom of the first vertical compartment (e.g. into the bottom of the tube), to create a density difference in the fluid in the first compartment and second compartment to drive the siphon flow. Hence, preferably, the first compartment comprises at a bottom part supply means, e.g. feeding pipe, for a gas stream, said gas advantageously comprising O₂ used for passivation. Hence, said gas is the preferably used additional fluid, and is referred to as additional gas stream. The vertical divider is preferably a vertical annular divider, more preferably a vertical tubular divider (e.g. a vertical tube), with a liquid passageway through (with openings in the tubular wall) and/or underneath at a bottom part of the divider for flow of liquid from the bottom of the second compartment to the bottom of the first compartment. The vertical divider is e.g. a tube, and e.g. has a length of at least 0.5 m to provide for an advantageous degree of siphon flow.

The first compartment is for instance inside the tubular divider and the second compartment is between the tubular divider and the reactor wall. The first compartment is e.g. located eccentrically in the bottom part. The second vertical compartment will generally be an annulus around the first vertical compartment, i.e. the annular space between the tubular divider and the vertical wall of the reactor. Advantageously, the feed pipe for liquid is a straight vertical pipe passing through the second compartment. Preferably, the outlet of the feed pipe for the liquid is outside the vertical projection of the first vertical compartment. The horizontal spacing between the first vertical compartment and the outlet of the liquid feed pipe, provides the advantage that the liquid can easily flow down in the second compartment.

Preferably, the feed pipe for gas extends through the tubular divider, such that the first compartment is an annulus between the gas feed pipe and the tubular divider. Preferably, accordingly the supply means for the additional fluid, e.g. for the additional gas, is provided as an annulus around feed pipe for gas. In this way, advantageously no additional opening needs to be created in the wall/shell of the reactor for the modification. In practice, an inner tube can be inserted through the existing tube for gas.

Optionally, the modified reactor comprises a tray above the second vertical level, i.e. above the outlet of the feed pipe for gas (and preferably also the feed pipe for liquid), e.g. at most 1.0 m above said outlets, or more preferably at most 0.50 m above said outlet. Optionally, the modification involves removing trays as necessary to create pipe for the one or more extended feed pipes.

The tray preferably comprises a horizontal plate. Preferably, the tray comprises a downward extending rim that is a perimeter of a zone of the tray (plate), and the plate comprises openings in this zone. Preferably, the openings are small such that in operation a gas layer forms underneath the plate in this zone, with the downward rim as a boundary, and with the rim extending into the liquid in operation. In this way, the upward flowing liquid is diverted horizontally, under the tray, radially outward towards the wall of the reactor. Preferably, the tray and reactor wall provide liquid passageways for the liquid to flow upward beyond the tray, e.g. by spacing between the tray and the wall. The movement of liquid horizontally to the reactor wall, also contributes of the flow of the liquid to the second vertical compartment.

Advantageously, in the embodiments with supply of passivation gas to the reactor bottom, more preferably in combination with siphon flow to prevent stagnant liquid in the reactor bottom, an existing reactor made of urea-grade austenite steel can be used, without the need to install duplex stainless steel elements (thought the use of duplex stainless steel is possible). The process is e.g. operated with at 0.3-1.0 vol. % O₂ in the CO₂ feed for passivation, hence for example at about the same O₂ level as before the modification.

In a possible embodiment, the bottom reactor part is provided with a vertical divider to create a siphon flow, to create two vertical compartments in the bottom part that are substantially separated by the vertical divider, in particular a first compartment inside the vertical divider and a second compartment between the vertical divider and the reactor wall. The vertical divider separates these two compartments over a part of the length of the reactor, i.e. over a part of the reactor height. In operation, a siphon flow is obtained from the liquid feed pipe outlet downward into the bottom part through the second vertical compartment, and upward through the first vertical compartment (i.e. inside the vertical divider, which is preferably a tubular divider).

Preferably, gas is introduced by the gas feed pipe, in a vertical middle part of the annular or tubular vertical divider, which divider extends in vertical direction above and below the outlet of the gas feed pipe, and which vertical divider permits for flow of liquid from the second compartment into the first compartment, e.g. by openings in the liquid vertical divider or an annular opening between the vertical divider bottom and the reactor bottom. Preferably, a gas distributor, such as a horizontal tray, is arranged in the first compartment (in the tubular divider) above the gas feed pipe, and near the gas feed pipe.

In embodiments wherein only the gas feed pipe is extended to have an outlet at a higher vertical level, the liquid feed pipe outlet can remain at low elevation (small vertical distance to the reactor bottom) and can be used to supply oxygen-containing liquid (from the condenser) to the reactor bottom part.

In connection with the previously discussed embodiments and preferred features, an aspect of the invention pertains to a method of modifying an existing urea plant.

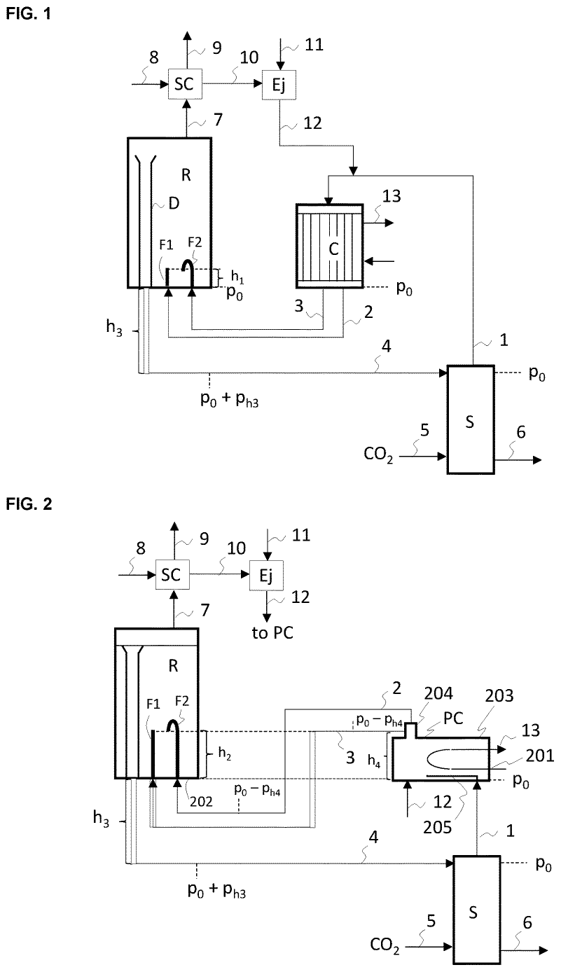

FIG. 1 schematically illustrates a non-limiting example of the existing plant that is modified in this method. References to FIG. 1 will be used in the following discussion without limiting to that embodiment.

The existing urea plant comprises a high-pressure (HP) synthesis section and at least one recovery section. The HP synthesis section is operated with high-pressure, i.e. absolute pressure of at least 100 bar, preferably at least 120 bar, and is used for reacting NH₃ and CO₂ to form urea. In the synthesis section, NH₃ and CO₂ are reacted under urea-forming conditions (at least 160° C. and a high-pressure, HP, of at least 100 bar) to form a urea synthesis solution comprising urea, water, ammonium carbamate (hereinafter: carbamate) and NH₃ and CO₂.

The HP synthesis section comprises a reactor (R), a stripper (S), and a HP carbamate condenser (C). The reactor is a vertical urea reactor, usually with a hemi head bottom and hemi head top (not shown) and a cylindrical shell. The urea reactor is provided with a downcomer/downpipe (D), to withdraw urea solution (4) from an upper part of the reactor and for flow downward of the liquid with a liquid column in the downpipe. The downpipe is e.g. provided as a vertical tube with a degassing funnel at the upper end. The downpipe passes through the bottom, e.g. through the hemi head bottom. In operation, a liquid level is maintained in the reactor and the funnel of the downpipe acts as an overflow weir with a liquid column maintained in the downpipe. Hence, in operation, non-condensed gas is separated from the liquid in an upper part of the reactor.

In an embodiment, the downpipe is provided with perforations in the bottom part to purge liquid from the bottom part of the reactor (not shown).

The reactor is provided with at least one fluid inlet (F1, F2)) at the bottom and a gas outlet (7) at the top. The reactor is preferably provided with a liquid inlet (F1) and a separate gas inlet (F2), both at the bottom. The liquid inlet and the gas inlet preferably each have a feed pipe which extends in the reactor. The gas inlet feed pipe is provided e.g. with a gooseneck, e.g. at 180° pipe fitting with gas flow downward in the downstream leg of the gooseneck.

The vertical position of the outlet opening of the gas feed pipe defines the first vertical height (h1).

The height h1 is e.g. 0.10 to 1.0 m for practical reasons and proper flow, typically 0.10 to 0.50 m.

The feed pipes have their openings at preferably the same height (h1) in the reactor, i.e. with the same vertical level for the outlet of the gas feed pipe and the outlet of the liquid feed pipe, and with the same vertical distance of said outlets to the top opening of the downpipe.

The vertical urea rector preferably comprises trays, e.g. horizontal trays with perforations. Preferably, the bottom tray is arranged vertically above the gas and liquid feed pipes.

The existing plant (and modified plant) comprises a HP stripper (S), which is a falling-film heat exchanger with a vertical tube bundle, configured to receive urea solution (4) from the reactor (in particular, from the downpipe) at a top inlet into the tube bundle. The urea solution (4) contains unconverted carbamate. The stripper has a gas top outlet (1), a liquid outlet (6) for urea solution from the bottom of the tube bundle, and has an inlet (5) to receive CO₂ used as strip gas at the bottom of the tube bundle. In the existing plant, preferably all of the gaseous CO₂ feed of the synthesis section is used as strip gas in the stripper. A heating fluid such as steam is used on the shell-side. The heating and strip gas promote the dissociation of carbamate in the urea solution in the tubes and the release of NH₃ and CO₂ in the gas stream (1). The liquid inlet of the stripper is spaced vertically lower than the bottom of the reactor (i.e. with a vertical spacing between the reactor bottom and the liquid inlet at the top of the stripper) and in operation a liquid column (h3) is maintained in the liquid flow line for urea solution from the downpipe to the stripper. This liquid column contributes to gravity flow of the urea solution (4) into the stripper. The stripper is suitably placed at ground level of the plant.

The liquid outlet of the stripper is connected to a recovery section, in particular to supply urea solution to a decomposer of a recovery section. Gas from the decomposer is condensed in a condenser to form a carbamate solution, which is recycled back to the synthesis section, preferably to the condenser, more preferably through the scrubber.

The HP carbamate condenser is connected to receive gas (1) from the stripper, preferably all of the gas from the stripper in the existing plant; this gas comprises NH₃ and CO₂. In the existing plant, the HP carbamate condenser is a falling-film condenser, i.e. a shell-and-tube heat exchanger with a vertical tube bundle, with gas to be condensed supplied to the top end of the tube bundle and liquid and non-condensed gas being withdrawn from the bottom end of the tube bundle. The liquid (2), i.e. the condensate, is a carbamate stream and is supplied to the reactor. In the existing plant, the vertical tube bundle is usually a straight tube bundle. A cooling fluid is provided in the shell, e.g. steam is raised in the shell. In the existing plant, usually the non-condensed gas (3) from the condenser bottom is also supplied to the reactor inlet, including passivation air comprised in the CO₂ feed used as strip gas in the stripper. The gas (3) contains NH₃ and CO₂ and condensation thereof in the reactor provides heat to drive the endothermic urea formation reaction in the reactor (R).

In the shell-side space of the HP carbamate condenser, steam (13) is raised from boiler feed water.

The bottom of the condenser is placed vertically higher than the liquid inlet of the stripper, with a sufficient vertical spacing to provide for a driving force of the gravity flow.

The synthesis section of the existing plant preferably comprises a HP scrubber (SC) which receives off-gas (7) from the gas outlet at the top of the reactor. This off-gas comprises NH₃, CO₂ and inerts; the inerts include at least O₂ used for passivation of the synthesis section, and usually also the N₂ comprised in the air used for passivation. The passivation air is added to the CO₂ feed in operation, in particular to the CO₂ feed to the stripper. The existing plant is for instance operated with adding air to the CO₂ feed in an amount corresponding to at least 0.3 vol. % O₂ in the CO₂ feed. In addition, the NH₃ feed and CO₂ feed may also contain inert components, such as Ar and N₂. The scrubber also receives a scrub liquid (8), in particular aqueous scrub liquid, more in particular carbamate solution from a recovery section. The scrubber is configured for gas/liquid contacting, to scrub CO₂ and NH₃ from the gas thereby forming a carbamate solution, and has a gas outlet (9) and a liquid outlet (10) for carbamate solution.

The existing plant also comprises a supply line (11) for NH₃ feed to the synthesis section, in particular to the existing condenser, preferably through an ejector (Ej). In the modification of the plant, this supply line is modified to be connected to the replacement condenser. This ejector is driven by said NH₃ feed and is used for transporting the carbamate solution (10) from the scrubber to the condenser. The ejector is for instance used if the scrubber is located at a lower elevation of the plant than the top of the reactor.

The combined stream (12) from the ejector is supplied to the condenser. It may be observed that the gas pressure at the reactor top is lower than in the condenser due to the liquid column in the process side of the reactor. It is also observed that this ejector (Ej) is a liquid-liquid ejector that is used for a relatively small carbamate stream from the scrubber and is not part of the main loop of the synthesis section. The main loop of the synthesis section is provided by the reactor, condenser, and stripper, and the gas line (1) from the stripper to the condenser, the liquid and gas flow lines (2,3) from the condenser to the reactor, and the liquid flow line (4) from the reactor to the stripper.

In an embodiment, liquid is purged from the bottom part of the reactor and supplied to this ejector (not shown).

As shown in FIG. 1, with a fixed pressure po at the gas outlet (1) of the stripper, which is also the pressure at the outlet of the condenser, the pressure of the urea solution at the inlet (4) is higher (h₃) because the reactor bottom is higher than the stripper top and a liquid column is maintained in the flow line from reactor bottom outlet to the stripper inlet (as shown). A sufficient height h₃ of this liquid column is used, and sufficient vertical spacing between stripper and reactor, to compensate dynamic losses in the main fluid loop of the synthesis section. The bottom of the condenser is located at substantially the same height as the bottom of the reactor. A sufficient vertical distance between the top of the stripper and the bottom of the condenser is maintained to ensure the gravity flow of the liquid in the synthesis section from the condenser, through the reactor, to the stripper.

FIG. 2 schematically illustrates a non-limiting example of the modified plant. Reference numerals are the same as in FIG. 1 unless specified otherwise. References to FIG. 2 will be used in the following discussion without limiting to that embodiment. Hence, the invention pertains to a method of modifying an existing plant to obtain the modified plant, and to the modified plant as such (e.g. also as newly built plant), and to a urea production process carried out in the modified plant.

In the modified plant, the falling film HP carbamate condenser is replaced by a replacement HP carbamate condenser (PC) which is a horizontal submerged carbamate condenser, preferably a pool condenser. The replacement condenser is a shell-and-tube heat exchanger with a tube bundle (201) and a shell (203). The tube bundle is a horizontal tube bundle, preferably a U-shaped tube bundle. The replacement condenser is operated with condensation of the gas from the stripper in the shell side space of the shell (203) and with a cooling fluid in the tubes. The shell (203) is typically a horizontal vessel, e.g. cylindrical with a hemi head and a tube sheet at horizontally opposed ends, with a horizontal length (i.e. from hemi head to tubesheet, along the tube bundle) and with a diameter; the length is usually larger than the diameter.

The cooling fluid is e.g. boiler feed water used for raising steam (13) in the tubes. Two or more types of cooling fluid can also be used, in respective tube bundles.

The carbamate condenser comprises a tube bundle (201), preferably a U-shaped tube bundle, with horizontal legs of the tubes in case of U-shaped tube bundle. The diameter of the condenser (vessel) is e.g. at least 2 m. In operation, the liquid layer in the condenser is e.g. in the range of 2 to 4 m. The condenser vessel is typically cylindrical.

The plant comprises e.g. a steam drum connected to the tube bundle for separating steam from liquid. The liquid, i.e. boiler feed water, is typically returned to the tube bundle.

The tube bundle is in operation submerged by liquid contained in the shell side space. In other words, the liquid level in the shell side space of the carbamate condenser is located above the upper tubes of the tube bundle. Hence, all tubes of the tube bundle are submerged in operation. In case of a horizontal U-shaped tube bundle, all straight legs (arranged horizontally) are submerged in the liquid in operation.

The liquid level can be maintained in the horizontal cylindrical vessel of HP carbamate condenser, or in the dome (204) provided on top of that vessel.

The gas (1) from the stripper is introduced into the pool condenser from the bottom, typically with a sparger (205) for distributing the gas horizontally. The sparger is mounted (is located) at least in part below the tube bundle. The replacement condenser hence comprises a sparger in the shell-side space, in particular at a bottom thereof, wherein the sparger receives gas from the stripper. The sparger usually is a manifold with a large number of exit openings for gas at the top, which exit openings are distributed in the horizontal plane and over the length of the vessel. Optionally, this sparger extends beyond the bend of the tube bundle in case of a U-shaped tube bundle. Thereby better use is made of the volume of the shell side space of the replacement condenser to provide residence time and urea formation can be obtained in the replacement HP carbamate condenser.

Carbamate solution (12) from the ejector is typically separately introduced into the condenser. Generally, carbamate solution originating directly or indirectly from a carbamate condenser of a recovery section of the plant is supplied to the shell side space, e.g. through the scrubber and ejector.

Generally, a part or all of the NH₃ feed of the plant is also supplied, as liquid, to the shell side space, e.g. combined with the carbamate solution. A second sparger in the shell side space can be used for distributing the NH₃ feed. This second sparge preferably extends only below the tube bundle.

The horizontal submerged condenser is typically a so-called pool condenser. The condenser has a fluid outlet at the top, in particular a gas outlet and a liquid outlet. The liquid outlet is arranged vertically above the tube bundle. In operation, a liquid layer with height h₄ is maintained in the horizontal submerged condenser, with a height larger than the diameter (in particular, the height) of the tube bundle, typically this height h₄ is at least the diameter of the condenser. Hence, the pressure of the gas (2) at the gas outlet is (p₀-ph₄). The condenser is operated with a liquid level in the condenser below this gas outlet.

By the extension of the gas feed pipe to a height h₂, e.g. to same height h₄, and maintaining the liquid level at the top of the reactor at constant vertical position, the gas can be supplied into the reactor. In other words, the outlet of the gas feed pipe is at a position in the reactor where the liquid pressure is (p₀-ph₄) or less. Herein the larger height h₂ indicates a smaller vertical distance to the upper end of the downpipe than in the existing plant. In an embodiment, only the gas feed pipe is extended. Preferably, the liquid feed pipe is also extended (as illustrated), to have a robust design wherein the liquid and the gas are introduced from the respective feed pipes into the reactor at the same vertical level in the reactor and hence at the same pressure.

Preferably, the position of the reactor is not changed during the revamp, in particular the vertical level of the reactor bottom is not changed.

The gas (2) is released from the second feed pipe (F2) into the liquid phase in the reactor, from the gooseneck, at the height h₂ and therefore into liquid in the reactor.

The diameter of the reactor is usually substantially the same as the diameter of the horizontal condenser (e.g. 3 m) and the volume of the horizontal condenser (PC) is larger than the bottom part of the reactor defined by the height h₂.

In the synthesis section of the modified plant, the scrubber is preferably used, as well as the ejector for transporting carbamate solution from the scrubber to the condenser.

In the modified plant, the reactor is for example provided with a drain for urea solution from the bottom, said drain connected to the urea solution inlet of the stripper, as discussed hereinbefore. In the modified plant, the bottom compartment of the reactor is e.g. provided with a lining of duplex stainless steel.

In a preferred embodiment, the gas feed pipe is extended in the modified plant, and further preferred also the liquid feed pipe.

Preferably, the distance from the second vertical level (h2) to the bottom of the reactor is at least equal to (equal to or larger than) the height of said tube bundle. It is noted that the height of the tube bundle is smaller than the height of the vessel.

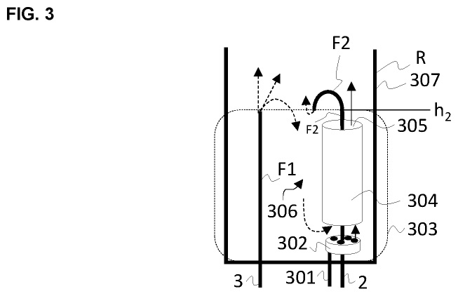

FIG. 3 schematically illustrates a non-limiting example of a preferred embodiment of the modified plant. Reference numerals are the same as in FIG. 2 unless specified otherwise. Dashed lines indicate flow patterns of the fluids. References to FIG. 3 will be used in the following discussion without limiting to that embodiment.

Preferably, the reactor of the modified plant comprises an inlet (301) for a fluid in a bottom part, in particular a third inlet. Preferably, this inlet is provided with a sparger (302) for horizontally distributing the fluid. Preferably, the fluid of the third inlet is a gas. Preferably, the third fluid comprises O₂ and is used for passivating the bottom compartment (303) of the reactor between the bottom (usually a hemi head) and the second height h₂ where the outlet openings of the gas feed pipe (F2) is located. The supply of fluid, preferably gas, comprising O₂ to this bottom compartment thereby helps to prevent corrosion of the bottom compartment.

As a further preference, the bottom compartment of the reactor comprises, below the second vertical level h₂ a vertical divider (304) which provides a confinement for gas from the third inlet (301), in particular from the sparger (302), to flow up. As discussed, the vertical divider in particular separates a first compartment (305) inside the vertical divider from a second compartment (305) between the vertical divider and the reactor wall (307) or shell.

Thereby, the fluid inside the vertical divider, i.e. in the second compartment (306) has a relatively low density. The vertical divider provides an aperture for liquid to enter, e.g. an aperture between the sparger and the vertical divider, or an opening in the vertical divider. The relatively lower density of the fluid inside vertical divider thereby draws in liquid, which results in flow of the liquid down from the outlet of the first feed pipe into a bottom end of said vertical divider. This provides for motion of liquid in the second compartment (306) of the bottom compartment, which ensures passivation of the reactor wall (exposed to said second compartment) and helps to prevent corrosion by stagnant carbamate-containing liquid.

Preferably, the vertical divider is arranged such that substantially all of the gas from the third inlet flows into the first compartment (305), e.g. the first compartment (305) is located straight above the sparger (302) for the gas from the third inlet. Preferably, a gap is provided between the bottom of the vertical divider and said spacing for liquid from the second compartment to enter the first compartment selectively at the bottom of the vertical divider; preferably the vertical divider is a tube without perforations in the tube wall.

Preferably, a feed pipe extends through the first compartment, preferably the gas feed pipe, to provide for a relatively higher gas content and lower density of the fluid in the first compartment. For instance, the first compartment is a relatively thin annulus around the gas feed pipe.

The pipe providing the inlet (301) for a fluid in a bottom part, and the sparger (302), can also be used for draining liquid from the bottom part of the reactor. The liquid can be supplied e.g. to the ejector (Ej). In a possible embodiment, the bottom part of the reactor is drained for liquid, e.g. to the stripper or to the ejector of the scrubber, with or without the introduction of passivating agent in the bottom part. The draining can help to avoid the formation of stagnant oxygen-depleted carbamate-containing liquid in the bottom part of the reactor.

The method of modifying an existing urea plant starts preferably from the existing plant of FIG. 1, and involves adding the replacement HP carbamate condenser, which is a horizontal submerged condenser, preferably a pool condenser, as discussed herein before, and modifying the reactor by extending the gas feed pipe, preferably both feed pipes. Preferably, the third inlet is added to the reactor, and preferably the vertical divider is installed as well in the method of modifying the existing plant. Suitably, the HP scrubber is not modified during said modification.

The invention also provides the urea production process carried out in the inventive urea plant, the process comprising supplying CO₂ feed to the stripper and NH₃ to the high-pressure carbamate condenser, which is the horizontal submerged condenser, preferably the pool condenser. The process involves operating the high-pressure synthesis section under a pressure of at least 100 bar to form urea, typically at a temperature of at least 160° C. In the process, the flow of liquid from the condenser to the reactor and from the reactor to the stripper is gravity flow, i.e. without the use of pumps or ejectors. Preferably, at least 20% of the urea is formed in the pool condenser, relative to total urea formed in the synthesis section. Suitably, passivation air is added to the CO₂ feed in an amount of least 0.3 vol. % O₂, relative to CO₂ feed.

The present disclosure also provides as an embodiment a method of modifying the existing urea production plant by changing the vertical location (elevation) of the reactor. The existing urea plant (A) comprises a high-pressure (HP) synthesis section comprising: a vertical urea reactor; a stripper having an inlet for gaseous CO₂ feed; a falling-film high-pressure carbamate condenser configured to receive gas rom the stripper, wherein the falling-film high-pressure carbamate condenser is a falling-film shell-and-tube heat exchanger having a bottom outlet from the tubes connected to a bottom inlet of the reactor, wherein the reactor optionally comprises one or more feed pipes for fluid from the condenser, the one or more feed pipes having an outlet at a first vertical level in the reactor. In the existing plant, the reactor is provided with fluid inlets(s) at the bottom, and the falling-film type carbamate condenser with fluid outlet(s) at the bottom, and both bottoms are placed at (substantially) the same elevation (vertical level). The modification involves: replacing the existing falling-film HP carbamate condenser with a horizontal submerged condenser, moving the entire vertical reactor upwards, in combination with the installation of a pool condenser, and the upward extension of the flow line from the reactor to the stripper at the upstream end. In this way, a lower pressure can be maintained in the top of the reactor, i.e. at the gas-liquid interface at the top of the reactor, whereas supply of urea solution from the reactor bottom into the HP stripper by gravity flow is still possible. However, arranging the vertical reactor at a higher level in the plant requires significant modifications of the plant, e.g. in terms of the support structure of the reactor and its connections, and this method of modifying the plant is a less preferred embodiment. Details and preferences for the existing plant and the horizontal submerged condenser are the same as for the embodiments with the modification of the feed pipes inside the vertical urea reactor.

The present disclosure also provides as an embodiment a method of modifying the existing urea production plant. Details for the existing plant are as for the existing plant (A). The method involves modifying the plant by cutting off a bottom part of the reactor, installing a new bottom plate at about the vertical level of the top of the horizontal submerged condenser, and supplying the gas and liquid from the horizontal submerged condenser to the new bottom plate; and replacing the existing falling-film HP carbamate condenser with a horizontal submerged condense. This embodiment is less preferred because it involves a loss of reactor volume in the reactor, and the method involves significant modifications of the construction of the reactor. Details and preferences for the existing plant and the horizontal submerged condenser are the same as for the embodiments with the modification of the feed pipes inside the vertical urea reactor.

The present disclosure also provides as an embodiment a method of modifying the existing urea production plant. Details for the existing plant are as for the existing plant (A). The method involves sealing off the bottom part of the existing vertical urea reactor, e.g. with a plate, replacing the existing HP falling-film carbamate condenser with the horizontal submerged condenser, and providing fluid lines for the gas and liquid from the horizontal submerged condenser to supply these fluids the bottom of the upper part of the reactor. This embodiment is less preferred because it involves a loss of effective reactor volume in the reactor, and, independently, the method may involve significant modifications of the construction of the reactor. Details and preferences for the existing plant and the horizontal submerged condenser are the same as for the embodiments with the modification of the feed pipes inside the vertical urea reactor.

The method of modifying an existing plant preferably gives an inventive plant. The inventive process is preferably carried out in the inventive plant. All preferences and details discussed in connection with the method of modifying an existing plant apply equally for the inventive plant, and all preferences and details for the inventive plant apply equally for the modified plant obtained with said method.

Carbamate, as used herein, refers to ammonium carbamate, as that term is used in the field of urea production.

The disclosure in summary pertains to a modification of a stripping-type urea plant by replacing the falling-film type HP carbamate condenser with a horizontal submerged condenser in combination with modification of the reactor internals, thereby permitting to maintain gravity flow in the modified plant.

1. A method of modifying an existing urea plant, wherein the existing urea plant comprises a high-pressure (HP) synthesis section comprising:
a vertical urea reactor;
a stripper having an inlet for gaseous CO₂ feed;
a falling-film high-pressure carbamate condenser configured to receive gas from the stripper, wherein the falling-film high-pressure carbamate condenser is a falling-film shell-and-tube heat exchanger having a bottom outlet from the tubes connected to a bottom inlet of the reactor,
wherein the reactor comprises one or more feed pipes for fluid from the condenser, the one or more feed pipes having an outlet at a first vertical level in the reactor;
wherein the method involves:
replacing the high-pressure carbamate condenser by a replacement carbamate condenser that is a horizontal submerged condenser which is a shell-and-tube heat exchanger with a tube bundle and with a shell side space, configured for condensation in the shell side space, preferably wherein the replacement condenser is a pool condenser; and
modifying at least one of the one or more feed pipes of the reactor to have an outlet at a second vertical level that is higher than the first vertical level, preferably modifying the one or more feed pipes of the reactor to have an outlet at a second vertical level that is higher than the first vertical level.

2. The method according to claim 1, wherein modified plant has a liquid flow line for condensate from the condenser to a first feed pipe of the reactor and a separate gas flow line from the condenser to a second feed pipe of the reactor, and the method involves modifying the second feed pipe to have an outlet at the second vertical level that is higher than the first vertical level.

3. The method according to claim 1, wherein the replacement carbamate condenser has a horizontal tube bundle, preferably a U-shaped tube bundle, and wherein the distance from the second vertical level to the bottom of the reactor is at least equal to (equal to or larger than) the height of said tube bundle.

4. The method according to claim 1, wherein the method involves providing the reactor with an inlet for an oxygen-containing fluid in a bottom reactor compartment that is provided between the reactor bottom and the second vertical level.

5. The method according to claim 4, wherein the method involves providing the reactor with a vertical divider in the bottom reactor compartment, wherein the vertical divider separates a first compartment from a second compartment between the vertical divider and the reactor wall, and wherein the vertical divider is arranged for receiving said oxygen-containing fluid and liquid from said second compartment into said first compartment at a bottom of said vertical divider.

6. The method according to claim 5, wherein said vertical divider is a vertical tubular divider.

7. The method according to claim 5, wherein a feed pipe extends through said first compartment, preferably wherein said second feed pipe extends through the vertical tubular divider.

8. The method according to claim 1, wherein the first vertical level is the same as the vertical level of the bottom outlet of the falling-film HP carbamate condenser.

9. The method according to claim 1, wherein the first vertical level is the same as the bottom of the shell side space of the replacement carbamate condenser.

10. The method according to claim 1, wherein the second vertical level is at a higher elevation than the upper tubes of the tube bundle of the horizontal submerged condenser.

11. A urea plant comprising a high-pressure synthesis section comprising:
a vertical urea reactor comprising a first and a second feed pipe;
a stripper having an inlet for gaseous CO₂ feed;
a high-pressure carbamate condenser configured to receive gas from the stripper, wherein the high-pressure carbamate condenser is a horizontal submerged shell-and-tube heat exchanger with condensation of gas from the stripper in the shell, having a horizontal tube bundle, preferably wherein the high-pressure carbamate condenser is a pool condenser,
and a liquid flow line for condensate from the condenser to a first feed pipe of the reactor and a separate gas flow line from the condenser to a second feed pipe of the reactor; wherein the vertical distance between the outlet of the second feed pipe for gas to the bottom of the reactor is at least equal to (equal to or larger than) the height or diameter of said tube bundle.

12. A urea plant according to claim 11, wherein the outlet of the second feed pipe is located vertically at the same height, or higher than, the liquid outlet of the high-pressure carbamate condenser; and preferably the outlet of the first feed pipe as well.

13. A urea plant according to claim 11, wherein the bottom of the high-pressure carbamate condenser and the bottom of the reactor are at the same vertical level of the urea plant.

14. A urea plant according to claim 11, wherein the reactor comprises an inlet for an oxygen-containing fluid in a bottom reactor compartment that is provided between the reactor bottom and the second vertical level.

15. A urea plant according to claim 11, wherein the reactor comprises internal supply means for introducing additional fluid into the reactor vertically between the bottom of the reactor and the point of introduction of the gas and liquid from the pool condenser.

16. A urea plant according to claim 11, wherein the reactor comprises a vertical divider in the bottom reactor compartment, wherein the vertical divider separates a first compartment from a second compartment between the vertical divider and the reactor wall, and wherein the vertical divider is arranged for receiving said oxygen-containing fluid and liquid from said second compartment into said first compartment at a bottom of said vertical divider;
preferably wherein said vertical divider is a vertical tubular divider.

17. A urea plant according to claim 16, wherein a feed pipe extends through said first compartment, preferably wherein said second feed pipe extends through said first compartment, preferably wherein said first compartment is provided by the inner space of the vertical tubular divider.

18. A urea production plant according to claim 11, wherein the horizontal submerged shell-and-tube heat exchanger comprises a capped horizontal cylindrical vessel holding the horizontal tube bundle, and the vertical distance between the outlet of the second feed pipe for gas to the bottom of the reactor is equal to or larger than the diameter of said horizontal cylindrical vessel.

19. A urea production process carried out in a plant according to claim 11, comprising supplying CO₂ feed to said stripper and NH₃ to said high-pressure carbamate condenser, and operating the high-pressure synthesis section under a pressure of at least 100 bar to form urea.

20. A urea production process according to claim 19, wherein the flow of liquid from the condenser to the reactor and from the reactor to the stripper is gravity flow.

21. (canceled)

22. (canceled)

23. (canceled)

24. (canceled)
